(19) United States
(12) Patent Application Publication
(10) Pub. No.: US 2006/0217815 A1
(43) Pub. Date: Sep. 28, 2006

(54) Modular prosthetic head having a flat portion to be implanted into a constrained liner

(75) Inventors: Phillip M. Gibbs, Winona Lake, IN (US); W. Jason Slone, Silver Lake, IN (US); Troy W. Hershberger, Winona Lake, IN (US); Kim S. Parcher, Etna Green, IN (US)

Correspondence Address: HARNESS, DICKEY & PIERCE, P.L.C., P.O. BOX 828, BLOOMFIELD HILLS, MI, 48303, US

(73) Assignee: Biomet Manufacturing Corp, Warsaw, IN (US)

(21) Appl. No.: 11/444,269

(22) Filed: May 31, 2006

Related U.S. Application Data
(63) Continuation-in-part of application No. 10/666,168, filed on Sep. 19, 2003.
(60) Provisional application No. 60/413,239, filed on Sep. 24, 2002.

(51) Int. Cl. A61F 2/32; A61F 2/30

(52) U.S. Cl. 623/22.17; 623/23.4

(57) ABSTRACT

A constraining prosthesis to reduce the possibility of a dislocation of the prosthesis after implantation is taught. A constraining liner is taught to include a constraining passage smaller than an internal diameter of the liner. A head can include a portion that is small enough to be positioned into the liner. The head can include a second dimension that is greater than the opening to constrain the modular head within the liner. A kit is also taught for trialing prosthesis to provide and determine a customized and personal fit of the prosthesis for each patient.

### CROSS-REFERENCE TO RELATED APPLICATIONS

This application is a continuation-in-part of U.S. patent application Ser. No. 10/666,168 filed on Sep. 19, 2003, which claims the benefit of U.S. Provisional Application No. 60/413,239, filed on Sep. 24, 2002. The disclosures of the above applications are incorporated herein by reference.

### FIELD

The disclosure relates to joint prosthetics, and particularly to ball and socket joint prosthetics forming a constrained joint.

### BACKGROUND

The human body includes many mobile or articulating joints. These joints allow at least two portions of bone to move relative one another when acted upon by a force. The joints also act to keep the bones aligned, so that proper and normal functioning of the joint may occur. Nevertheless, during a lifetime different joints may become injured or affected by wear. When this occurs, the joint no longer operates as it should. The bone may easily move out of alignment or not retain its natural operating orientations. In addition, soft tissue holds the joints in place. Likewise, during a lifetime of use and wear the soft tissue may become injured or damaged, so that it no longer holds the joint as it should. When this occurs, one common effect is that the joint easily becomes dislocated. Multiple dislocations can further injure the joint and reduce the ability of the soft tissue to hold the joint in the proper orientation. At this point, a reconstruction of the joint most often is performed.

Several types of joint prosthetics are generally known in the art. A constrained prosthetic may be used when dislocation is a constant or repeated issue. The constrained prosthetic provides a ball and prosthetic socket where the ball of the prosthetic is held within the prosthetic socket or an internal cavity of the prosthetic by a mechanical means. For example, a metal ring may be placed around the opening of a liner portion disposed in the prosthetic socket to hold the ball of the joint prosthetic within the liner portion. The ring increases the lever out force needed to remove the ball from the liner portion. This makes dislocation of the ball portion from the liner portion less likely. The ring may either be assembled onto the liner portion during the manufacturing process or it may be installed during the operative procedure. Generally, however, if the ring is to be installed during the operative procedure, the liner portion must include deflectable portions separated by slits to allow the physician to install the ring.

It is also known to provide a liner that includes an internal diameter or cord that is greater than its opening. Specifically the ball portion of the joint prosthetic is keyed to the liner opening. Therefore the ball portion may be implanted in only one orientation. This allows for placement of the ball portion but resists its dislocation. The implantation orientation generally is not the natural orientation of the joint, where the ball is able to be inserted into the shell portion. Then, when the joint is moved to the natural position the ball is not easily removed from the shell. These prosthetics do not include other portions, such as a ring, which increase the lever out force of the prosthetic.

With these generally known constrained joint prosthetics, if a dislocation occurs, regardless of the constrained liner, closed reduction of the joint is generally difficult without an operative procedure. Because the constrained joint includes a portion which substantially closes the interior shell area from the ball joint, it may be difficult to perform a closed reduction of the joint into its normal orientation. Therefore, it is desirable to provide a prosthetic joint, which will include the advantages of a constrained liner, but also allow for an easy reduction of the joint if a dislocation occurs after the operative procedure.

### SUMMARY

A joint prosthetic including a constrained shell liner to decrease the possibility of a post-operative dislocation. The constrained liner may be implanted into a prosthetic cup or into the bone itself. The ball portion of the joint being substantially spherical, but defining a cylinder around an equator, to allow implantation of the ball joint into the constrained liner. The equator defining the cylinder may be formed at any appropriate angle relative to an aperture defined by the ball, such as an aperture to receive a modular portion. The constrained liner includes an opening, into the interior or socket area. The opening or entrance has a smaller diameter or dimension than a diameter of the interior of the constrained shell. Nevertheless, the diameter of the cylinder equator of the ball is formed so that it is able to pass through the opening of the constrained liner. Generally, the cylindrical equator is placed on the ball portion of the joint, such that implantation occurs at a generally unnatural orientation of the ball portion to the shell portion of the prosthetic joint. Therefore, when the limb is placed in a natural orientation, the ball portion is substantially constrained within the constraining liner. A ring, formed of a material that is substantially rigid under normal anatomical conditions, such as titanium, may be placed around the opening of the constrained shell to increase the lever-out force required to dislocate the ball from the shell portion.

According to various embodiments a prosthetic joint for replacement of a natural joint that resists dislocation is taught. The prosthetic joint includes a liner having an internal concave portion defining a cup diameter, and defining an opening having a passage width smaller than the cup diameter. A modular ball portion has a ball diameter substantially equal to the cup diameter. A constraining ring cooperates with the opening to resist a removal of said ball portion from said liner after implantation. The ball portion includes an equator having a diameter similar to the passage width. The ball portion is adapted to be implanted into the internal concave portion during an operative procedure. The constraining ring is assembled prior to the operative procedure.

According to various embodiments a method of implanting a joint prosthesis having a modular stem portion and modular head portion to be associated with a constraining liner after an implantation thereof is taught. The method includes implanting a modular stem into a first boney portion and implanting a cup into a second boney portion. A trial liner is then temporarily associated with the cup. A proper modular head to operably associate with the modular stem and the cup after implantation is chosen. Choosing the proper modular head includes associating a trial modular head with the modular stem, disposing the trial modular head in the trial constraining liner, and moving the first boney portion through a range of motion while the trial modular head is in the trial constraining liner. Finally, the trial portions, including trial modular heads and trial liners, are replaced by implantable portions having the appropriate range of motion, as determined by the trial portions.

According to various embodiments a method for implanting a dislocation resistant joint prosthesis having a constraining liner affixed in a cup, and a modular head portion, extending from a modular stem member, implantable into the constraining liner is taught. The method includes implanting the cup into a first boney portion and affixing the constraining liner to the cup. A modular stem member is implanted into a second boney portion and a modular head portion is disposed on a neck of the modular stem member. The second boney portion is oriented in an unnatural orientation and the modular head portion is inserted into the constraining liner. The second boney portion is moved to a natural orientation after the head portion is implanted into the constraining liner.

According to various embodiments a kit to implant a modular hip joint is taught. The kit includes a modular stem to be implanted into a femur and a modular head adapted to extend from the modular stem. The modular head has a major diameter. A modular trial head is adapted to cooperate with the modular stem to select the modular head. An acetabular cup is implanted into an acetabulum and a constraining liner, defining an entrance, may be affixed into the acetabular cup. A trial liner cooperates with the acetabular cup and the modular trial head during a trialing process. The entrance has a dimension less than the major diameter of the modular head. The constraining liner and the modular head interact to resist a dislocation of the modular head from the constraining liner after implantation.

Further areas of applicability of the present teachings will become apparent from the detailed description provided hereinafter. It should be understood that the detailed description and specific examples, while indicating the preferred embodiment of the teachings, are intended for purposes of illustration only and are not intended to limit the scope of the teachings.

### BRIEF DESCRIPTION OF THE DRAWINGS

The present teachings will become more fully understood from the detailed description and the accompanying drawings, wherein:

FIG. 1 is an exploded perspective view of a constrained modular hip prosthetic according to a first embodiment;

FIG. 2 is a plan view of a kit for the implantation of the hip joint prosthetic;

FIG. 3 is a detailed perspective view of a method for trialing the hip prosthetic;

FIG. 4 is an exploded plan view of an implantation method of the hip prosthetic;

FIG. 5 is a detailed partial cross-section view of the hip prosthetic in its implanted and natural position;

FIG. 6 is an exploded perspective view of a constraining modular hip prosthetic according to a second embodiment;

FIG. 7A is a view of an implant portion that includes a cylindrical edge having a plane that is substantially parallel to a central axis of the implant portion;

FIG. 7B is a view of an implant portion having a cylindrical edge that is at an angle to a central axis of the implant portion;

FIG. 8 is an exploded detail view of a bi-polar implant according to various embodiments;

FIG. 9 is an environmental view of a bi-polar implant according to various embodiments;

FIG. 10 is an exploded view of a bi-polar implant according to various embodiments; and

FIG. 11 is an environmental view of a bi-polar implant according to various embodiments.

### DETAILED DESCRIPTION OF VARIOUS EMBODIMENTS

The following description of various embodiments is merely exemplary in nature and is in no way intended to limit the teachings, its application, or uses. Although the following description specifically relates to an acetabular prosthesis, including an acetabular constrained liner and a femoral head prosthetic, it will be understood that the present teachings may be used for any prosthetic joints requiring a ball portion and a socket portion. Moreover, although the following description relates to a constrained liner fixed in a prosthetic cup, the liner may also be implanted directly into a boney portion.

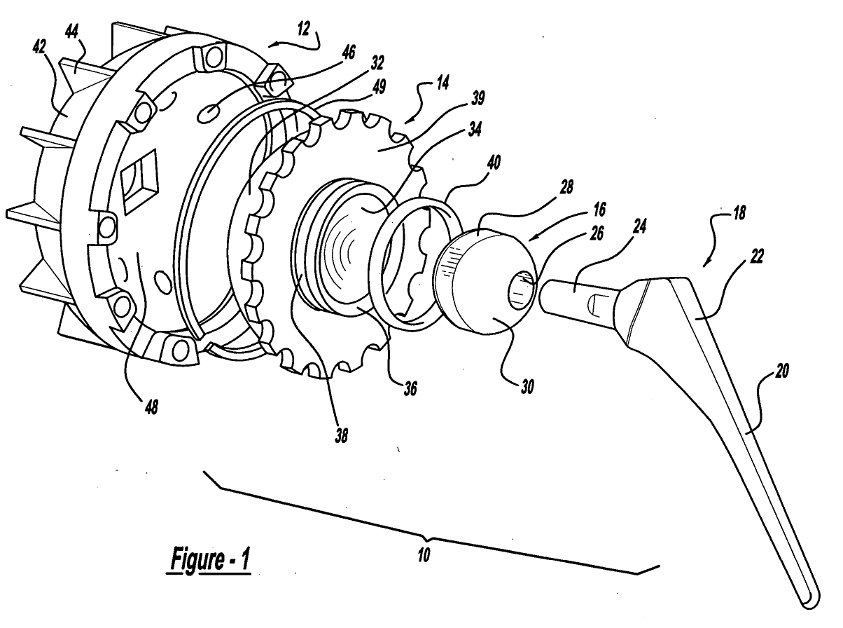

With reference to FIG. 1, a joint prosthetic 10 generally includes an acetabular cup or shell 12. An acetabular liner, that is substantially solid also referred to as a constrained liner 14, a femoral head prosthetic 16, and a femoral stem prosthetic 18. Although the following discussion refers to the constrained liner fixed within an acetabular cup 12, it will be understood that the constrained liner 14 may simply be fixed into a boney structure. The femoral stem 18 generally includes a stem portion 20, which is received into the femur during implantation. A shoulder or transition portion 22 is provided between the stem 20 and a neck 24. The neck 24 generally forms a Morse taper with a female taper 26 defined in the femoral head 16. The female taper 26 and the neck 24 form a substantially friction lock junction between the femoral stem 18 and the femoral head 16. It will be understood that a taper junction may also be formed when a-male taper extends from the femoral head 16 and a female taper is provided on the stem 18.

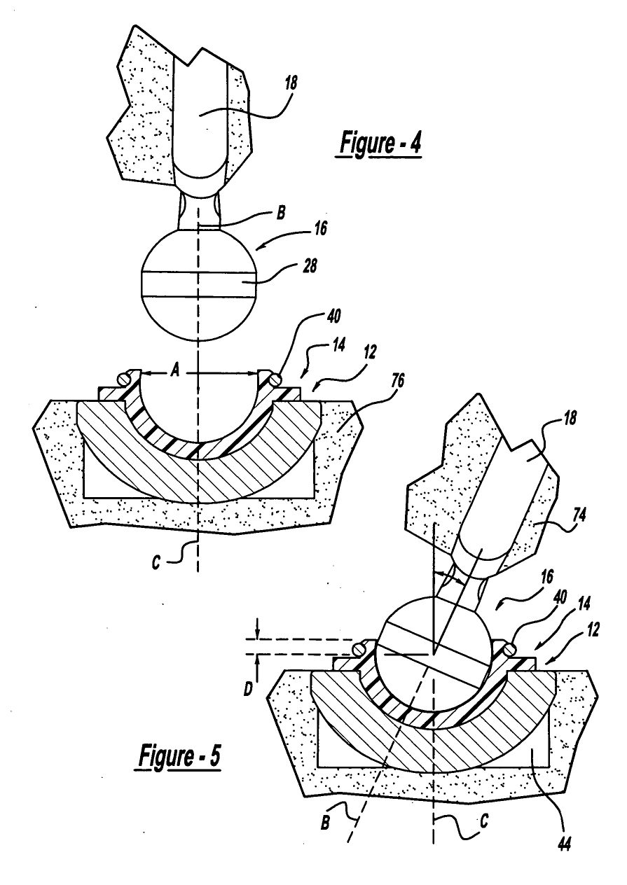

The femoral head 16 is substantially spherical. The sphere of the femoral head 16 is interrupted to allow the creation of the female taper 26 and a depressed or cylindrical equator 28. The cylindrical equator 28 is formed at an appropriate equator, as described herein, to allow for easy implantation of the femoral head 16 into the constrained liner 14, but does not allow for easy removal of the femoral head 16 from the constrained liner 14 when the modular stem 18, implanted in a femur, is in an anatomical position. The equator around which the depressed equator 28 is formed may be any equator and may be of an angle to a central axis of the femoral head 16. The cylindrical equator 28 is formed about an axis B (as shown in FIG. 4) which defines a center of an implantation presentation face.

The femoral head 16 includes an exterior surface 30, which is generally highly polished to a mirror-like finish, to allow easy articulation of the femoral head 16 within the constrained liner 14. The femoral head 16 includes a major or spherical diameter which is defined as a diameter between any two points on the exterior surface 30, of the femoral head 16, and passing through the spherical center of the femoral head 16. The femoral head 16 also defines a cylindrical or equator diameter. The equatorial diameter is defined as the diameter of the cylinder defining the cylindrical equator 28. The cylindrical equator 28 may be oriented anywhere on the femoral head 16. The cylindrical equator 28 may circle the center of the femoral head 16 or may be offset therefrom. Also, the cylindrical equator 28 may be placed at any orientation relative to the female taper 26. The equatorial diameter or distance is generally smaller than the spherical or major diameter of the femoral head 16.

The constrained liner 14 includes an exterior hemisphere 32 and an interior hemisphere or socket portion 34. On a distal side of the liner 14 is defined a liner projection 36. It will be understood that the exterior and the interior of the constrained liner 14 may nof be exactly a hemisphere. Extending between the projection 36 and the exterior of the liner 14 is a land area 39. The liner projection 36 is substantially annular and includes an annular track 38, defined on the exterior thereof. The annular track 38 receives a constraining ring 40 during production. It will be understood that the constraining ring 40 may also be installed in the annular track 38 intraoperatively. The constraining ring 40 may be placed into the annular track 38 using any appropriate means. For example, the ring 40 may be mechanically forced over the projection 36 and into the annular track 38. Nevertheless, the ring 40 is generally placed in the track 38 pre-operatively.

The acetabular cup 12 defines an exterior 42, which is placed in a prepared acetabulum. Extending from the exterior of the acetabular cup 12 are spikes or projections 44 that may be used to imbed the acetabular cup 12 into the acetabulum. In addition, a plurality of screw holes 46 are provided to allow screws to be placed through the acetabular cup 12 to be received in the acetabulum. It will be understood that only one or neither of the spikes 44 or the screw holes 46 may be provided, moreover the cup 42 may be fixed with a cement. The interior of the acetabular cup 12 defines a concave hemisphere portion 48. The diameter of the internal hemisphere 48 of the acetabular cup 12 is substantially equal to an exterior diameter of the exterior convex surface of the constrained liner 14. Therefore, the acetabular liner 14 is substantially received into the acetabular cup 12. If the constrained liner 14 is, however, not hemispherical, then the interior of the cup 12 will also not be a hemisphere. Any appropriate means may be used to secure the acetabular liner 14 into the acetabular cup 12. For example, a locking ring 49 or a bone cement may be used to fix the acetabular liner 14 into the acetabular cup 12. It will also be understood that an appropriate constrained liner 14 may not need to be implanted in the cup 12, but rather implanted directly into the acetabulum.

The appropriate size of the acetabular shell 12 may be determined either pre- or intra-operatively. Varying sizes of the acetabular cup 12 and the acetabular liner 14 may be provided to customize a fit for a particular patient. Nevertheless, a single size of the femoral head 16 may be provided because of the modular acetabular and femoral implant. Therefore, the interior hemisphere 34 of the acetabular liner 14 remains the same and similar to the exterior diameter of the femoral head 16. Nevertheless, it will be understood that a plurality of femoral head diameters may be provided which would be implanted with related sized constrained liners. Having a single size femoral head 16 can allow a reduction in inventory only requiring varying taper depths.

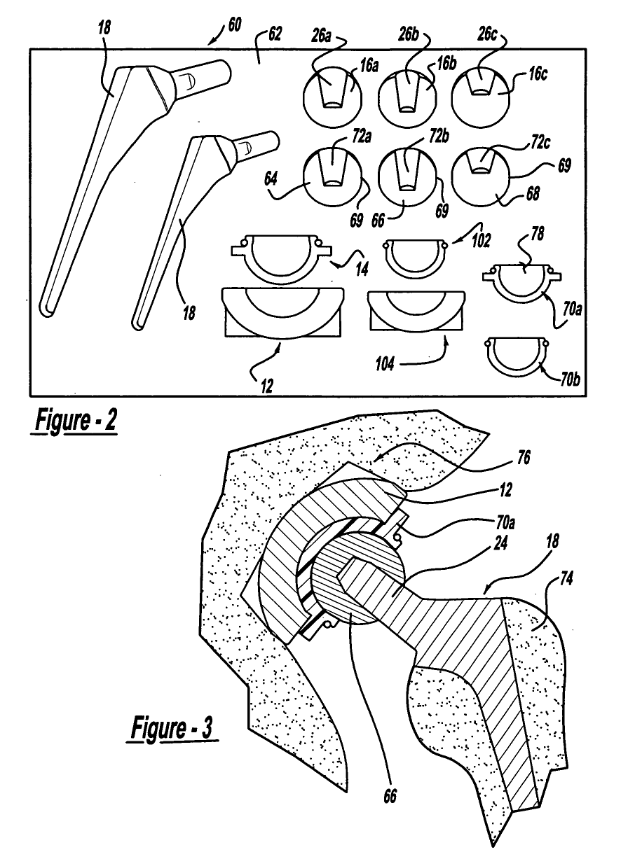

With reference to FIG. 2, a kit 60 allows for a substantial personalization or custom fit of the prosthetic 10 to the particular patient. As described herein, a trial and implantation method may be used in conjunction with the kit 60 to assure a most preferred fit of the joint prosthetic 10. This customization allows for a more precise and natural orientation of the femur to the pelvis. The kit 60 may include a container 62, which includes multiple femoral stems 18, the multiple acetabular liners 14, 102 and the multiple acetabular cups 12, 104. Also included in the kit 60 are a plurality or set of femoral trial heads 64, 66 and 68 each having a cylindrical diameter 69. It will be understood that although the set of femoral trial heads includes three any appropriate number of trial heads 64, 66, 68 may be included. Also included is a set of trial liners 70a, 70b, that correspond to one of the implant constraining liners 14, 102. Finally, a set of final or implantable femoral heads 16a, 16b, and 16c are provided in the kit 60. Although the kit 60 is here illustrated to include both the trial and implant portions, this is not intended to be a limiting example. For example, the kit 60 may only include the trial portions while the implant portions are kept separately, and only opened when the appropriate size has been selected. In this instance, the kit 60 would substantially be a trial kit and only include the trial heads 64, 66, 68 and the trial liners 70a and 70b.

Each of the implantable heads 16a, 16b, 16c includes a feature substantially equivalent to one of the trial femoral heads 64, 66, 68. Generally, the trial heads 64, 66, 68 have a trial female taper 72 that has an associated and varying depth. Each of the implantable femoral heads 16a, 16b, 16c includes a depth of the female taper 26a, 26b, 26c which is equal to one of the female tapers 72 of the trial heads 64, 66, 68. Therefore, the kit 60 allows for a trialing of the joint prosthetic 10 to insure a proper and customized fit. The varying depth of the female tapers 26a, 26b, 26c effectively varies the length of the neck 24, which varies the distance between the stem 18 and the constrained liner 14. The shallower the female taper 26 the further the stem 18 is held from the liner 14. This both aligns the femur and properly tensions the soft tissue.

With reference to FIGS. 2-5, one method for trialing and implanting the prosthetic joint 10, including the kit 60, includes implanting the femoral stem 18 into a femur 74. The femur 74 may be prepared to receive the femoral stem 18 using any appropriate and generally known method. Likewise, an acetabulum 76 is prepared to receive the acetabular cup 12. Methods of preparing the acetabulum 76 are also generally known in the art and any are appropriate to prepare the acetabulum 76 for the acetabular cup 12. After the femur 74 and the acetabulum 76 are prepared, the femoral stem 18 is implanted into the femur 74 and the acetabular cup 12 is implanted into the acetabulum 76. As discussed above, an appropriate liner may be provided which is implanted directly into the acetabulum 76.

During the trialing phase of the operative procedure, the trial liner 70 is temporarily placed into the acetabular cup 12. Although the trial liner 70 is illustrated to include a constraining ring in a constraining format, and may be so formed in the trial procedure, it is not necessary that the trial liner 70 be a constraining liner. Rather, the trial liner 70 may simply include a shape or size that substantially mimics the shape and size of the constraining implant liner. This may increase the ease of the trial process by not constraining the trial head within the trial liner and thus decreasing the time required for the trialing procedure. Therefore, it will be understood that the trialing liner 70 may either include a constraining portion or not include a constraining portion. The trial liner 70 may be held in place with a temporary cement or other appropriate temporary methods. For example, the ring lock 49 may be used to temporarily hold the trial liner 70 in place. The trial liner 70 has an opening or entrance 78 similar or the same as the opening in the acetabular liner 14. The cylindrical diameter 69 of the trial heads 64, 66, 68 is small enough to allow easy placement in and removal from the trial liner 70 while simulating the prosthetic. This allows for easy trialing of the various femoral trial heads 64, 66, 68 and for speed and efficiency of the trialing procedure. A trial constraining ring may also be included on the trial liner 70, to further simulate the constraining liner 14. The trial liner 70 is designed to simulate the constraining liner 14 for range of motion and positioning of the femur after implantation.

The physician may choose the first trial femoral head 64, which includes the female taper having a depth of about 3 mm. The physician may place the trial femoral head 64 onto the neck 24 of the femoral stem 18. The physician may then place the femoral stem 18, including the trial head 64 into the trial liner 70. The physician then determines whether an appropriate fit has been obtained. If not, the physician may then attempt to trial the second femoral head 66, including a female taper 72b, having a depth of approximately 2 mm. Again, the physician may test a range of motion and orientation using the second femoral trial head 66 by placing it into the trial liner 70.

The physician may trial each of the trial femoral heads 64, 66, 68 to determine the appropriate length of the female taper 26. The physician may also trial each of the trial liners 70a and 70b and trial acetabular cups. The trial liners 70a and 70b are placed in the acetabulum 76 or the cup 12 and the femoral head is placed therein and moved through a range of motion to check for early impingement. If early impingement is discovered, the physician may move the trial liner 70a, 70b or choose a different trial liner. In any case, the physician can trial for both liner impingement and proper soft tissue tightness with the trial liners 70a, 70b, and the trial heads 64, 66, 68. Therefore, a complete customization of the prosthetic implant 10 can be obtained. Although not illustrated, the liner 14, 102 need not include only a flat face liner. It may include an internal slant or a high wall. When the prosthetic liner to be implanted is not a flat face liner, such as one that includes a highwall or internal degree, the physician may also trial the position and orientation of the trial liner 70a, 70b. Nevertheless, due to the orientation and shape of the trial acetabular femoral heads 64, 66, 68 a trial can be performed before implanting the constrained liner 14 in its final orientation. Once the physician has determined the appropriate length of the female taper 26, the physician then chooses the appropriate femoral head 16a, 16b, or 16c. The femoral heads 16 include the female taper 26 of equivalent depth to one of the trial femoral heads 64, 66, 68. Therefore, an appropriate femoral head 16 cup 12 position and liner 14 position, may be chosen through the trialing procedure and the kit 60.

With reference to FIGS. 4 and 5, a method of implanting and reducing the joint prosthetic 10 is illustrated. Once the appropriate femoral head 16 has been chosen, it is placed on the neck 24 of the femoral stem 18, while the joint is dislocated. Also, the constrained liner 14 is affixed in the selected orientation into the acetabular cup 12. The constrained liner 14 already has installed the constraining ring 40 so that once the femoral head 16 is implanted into the constrained liner 14 the femoral head 16 cannot be easily removed therefrom. Moreover, because the constraining ring 40 is already installed on the constrained liner 14 the physician need not install the constraining ring 40 during the operative procedure. The cylindrical equator 28 of the femoral head 16 allows it to be implanted into the acetabular liner 14 with the constraining ring 40 in place. Nevertheless, as discussed above, the constraining ring 40 may be provided separately from the constrained liner 14 and the constrained ring 40 may be implanted intra-operatively by the physician.

The projection 36 of the acetabular liner 14 defines an opening or entrance diameter A (illustrated in FIG. 4). The opening diameter A is similar to the diameter of the cylindrical equator 28. The opening diameter A may be equal to, slightly smaller or larger than the diameter of the cylindrical equator 28. For example, if the diameter A has length of about 34 mm, the diameter of the cylindrical equator 28 may be about 34.2 mm. Therefore, with an acceptable amount of force the femoral head 16 may be pushed through the entrance defined by the projection 36 and into the concave interior 34 of the acetabular liner 14. Nevertheless, the distance A may be any appropriate length. This is done by aligning an axis B of the cylindrical equator 28 substantially coaxial with a central axis C of the acetabular liner 14. Although an axis B is illustrated to be generally aligned with the stem 18 it does not necessarily need to be so. For example, the axis B of the cylindrical portion 28 may be formed at an angle relative to the stem 18. Therefore, it will be understood that the axis B defined by the cylindrical portion 28 may be formed at any angle relative to the stem portion 18 or a portion of the head, such as the aperture 26 to receive a portion of the stem 18, at any appropriate angle and may be about 0 to about 180° relative thereto. When this occurs, an implantation face of the femoral head 16 is presented and is substantially equal to the diameter A of the opening of the liner 14. Therefore, the femoral head 16 may be received into the concave interior 34 of the acetabular liner 14. Generally, after the femoral head 16 has been implanted onto the neck 24 of the femoral stem 18, the axis B of the cylindrical equator 28 is coaxial with the axis C of the acetabular liner 14 when the femoral stem 18 and the femur are in an unnatural or non-anatomical position. Therefore, it is less likely that this orientation will occur after implantation is completed.

With reference to FIG. 5, once the femoral head 16 is implanted into the acetabular liner 14 and placed in an anatomical position, the axis B of the cylindrical equator 28 is no longer coaxial with the axis C of the acetabular liner 14. Essentially, the axis B and the axis C intersect when the stem 18 is placed in a natural or anatomical position. Therefore, because the diameter of the femoral head 16 is greater than the diameter A of the acetabular liner 14, the femoral head 16 may not easily dislocate from the acetabular liner 14 when the axes B and C intersect. In addition, the constraining ring 40 assists in making rigid and stronger the projection 36 surrounding the opening of the acetabular liner 14. Therefore, the rigidity of the acetabular liner 14, generally formed of a high molecular weight polyethylene, is reinforced by the constraining ring 40. Moreover, post operatively, the acetabular liner 14 extends a distance D above a hemisphere of the femoral head 16. The distance D is generally less than about 15 mm. This is necessary to provide a force, which will constrain the femoral head 16 within the acetabular liner 14 after implantation.

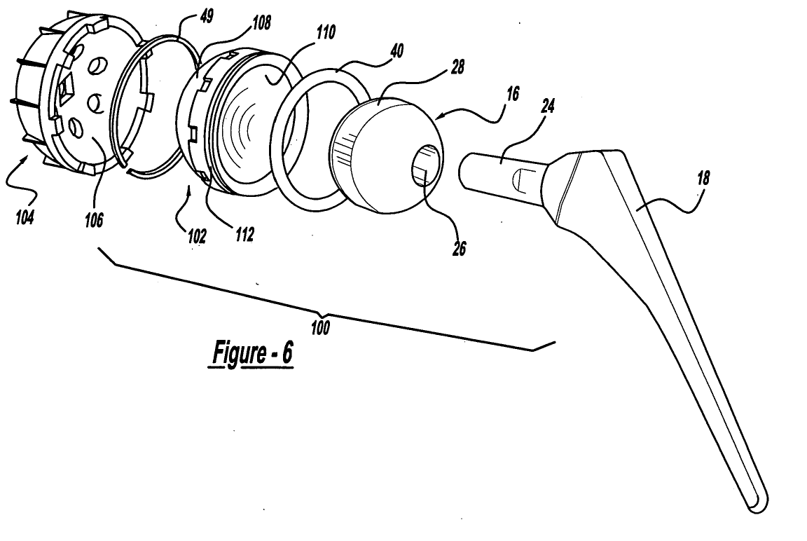

With reference to FIG. 6, a prosthetic joint 100 according to a second embodiment is illustrated. The prosthetic joint 100 includes the femoral stem 18 and the femoral head 16. The femoral head 16 again includes a female taper 26 and a cylindrical equator 28. Moreover, the prosthetic joint 100 includes the constraining ring 40, which is received on an acetabular liner 102. The acetabular liner 102 is received in an acetabular cup 104. The acetabular cup 104 includes an exterior, which has a diameter, which is smaller than the exterior diameter of the acetabular cup 12. Therefore, the acetabular cup 104 also includes a concave interior 106, which has a diameter smaller than the diameter of the concave interior of the acetabular cup 12. This, in turn, allows that the exterior diameter of the convex portion of the acetabular liner 102, to be smaller.

Due to this smaller size, the acetabular liner 102 does not include a land area similar to the land 39 of the acetabular liner 14. Nevertheless, the concave-interior 110 of the acetabular liner 102 can be substantially similar in size to the interior of the acetabular liner 14. Therefore, the single femoral head 16 may be used in any size acetabular cup 12, 104. In addition, a single constraining ring 40 can be received on a plurality of sizes of acetabular liners 14, 102. The acetabular liner 102 includes an annular track 112, which receives the constraining ring 40. According to this embodiment, the thickness of the walls of the acetabular liner 102 may be thinner than the walls of the larger acetabular liner 14, but are still appropriate for the implantation into the acetabular cup 104.

The cylindrical equator 28 allows the femoral head 16 to be inserted into the constraining liner 14, 102 regardless of the size of the femoral head 16. The femoral head 16, therefore, can be large enough to provide an optimum range of motion once implanted into the patient. Moreover, the constraining liner 14, 102 includes a high lever out strength due to the fact that the projection 36 is substantially solid and does not include any breaks or slots therein. Therefore, when a lever out force is applied through the femoral head 16, the force is distributed through a substantial portion or all of the projection 36 and the ring 40. Again, this allows for the use of larger diameters for the femoral head 16 without increasing the possibility of a dislocation due to a lever out of the femoral head 16 from the constraining liner 14, 102.

In conjunction with the substantially spherical femoral head 16, the distance D that the acetabular liner 14, 102 extends beyond the hemisphere of the femoral head 16 also allows a great range of motion. Rather than extending a longer distance above the hemisphere of the femoral heads 16, the shape of the femoral head 16 substantially holds the femoral head 16 in the acetabular liner 14, 102. The shape of the femoral heads 16 cooperate with the distance D to ensure that the femoral head 16 does not easily dislocate from the acetabular liner 14, 102. Essentially, the diameter of this sphere defined by the femoral head 16 is substantially equal to the diameter of the hemisphere defined by the acetabular liner 14, 102. Moreover, the diameter of the femoral head 16 is larger than the opening A of the acetabular liner 14, 102. It is only the inclusion of the cylindrical equator that allows for an implantation of the femoral head 16 into the acetabular liner 14, 102. The interaction of these features allows a range of motion, after implantation, of at least about 80°. Nevertheless, the distance D can be increased and still allow for a great range of motion because of the sizes of the femoral head 16 allowed due to the inclusion of the cylindrical equator 28. Therefore, a distance D that increases the lever out strength of the prosthetic 10, 100 will still allow for a large range of motion.

One measure of lever out force is the force generally created when the neck 24 of the stem 18 engages the projection 36 of the acetabular liner 14 and creates a force that attempts to dislocate the femoral head 16 from the internal cavity 34. This force is resisted by the interaction of the surface 30 of the femoral head 16 with the projection 36 which is reinforced by the constraining ring 40. It will be understood that other definitions of lever-out force or strength are generally known and may also be increased through use of the constrained liner 14, 102 and the femoral head 16.

Therefore, the joint prosthetic 10, 100 can be provided that includes a constrained liner to reduce the possibility of a dislocation post-operatively of the prosthetic joints 10, 100, which does not include the physician being required to do any more than implant the proper size of the acetabular cup 12, 104 and femoral head prosthetic 16. In addition, the implantation of the femoral head prosthetic 16 into the acetabular liner 14, 102 only requires the proper orientation of the femoral head 16. Therefore, excessive force is not required to implant the femoral head 16 into the acetabular liner 14, 102. Moreover, because the constraining ring is provided before the operative procedure begins, the steps required to implant the prosthetic joint, 10, 100 are reduced. Nevertheless, the advantages included in having the constraining ring 40 provided with the joint prosthetic 10, 100 are retained.

Moreover, if a post operative dislocation occurs, of the prosthetic joint 10, 100 a closed reduction of the prosthetic joint 10, 100 can occur without need for additional open surgery. This can be done by moving the femoral head 16 to the proper orientation, such that the axis B of the prosthetic femoral head 16 is aligned with the axis C of the acetabular liner 14, 102.

The cylindrical equator defined by the head portion that is generally held within the constraining liner may operably interact with the liner such that a non-sealing fit is formed with a portion of the liner during physical use. This allows a fluid, such as a natural or biological fluid, to flow between or through the head portion and the liner portion. The fluid may reduce friction between the head or ball portion and the liner portion after implantation. In addition, the presence of the fluid may increase lever out or pull out forces while reducing wear on the liner portion. This may increase the longevity of the implant and decrease the necessity for revision procedures. In addition, because of the cylindrical equator, the entire surface area of the sphere which is generally defined by the head portion or ball portion, does not contact the liner. That is because of the cylindrical equator or a portion of the entire sphere or arc of the head does not contact the liner at a given time. This may also reduce wear on the liner over a plurality of cycles after implantation of the implant. This may again increase longevity and reduce the possibility of a revision procedure due to wear on the liner.

It will also be understood that the head portion need not necessarily be used with a constraining liner. The head, with the cylindrical equator, may be used with any appropriate liner or acetabular implant. As discussed above the cylindrical equator may reduce wear and reduce the need for a revision procedure. Though this theory is not intended to limit the scope of the appended claims, but simply provides that the head need not only be used in a constrained liner.

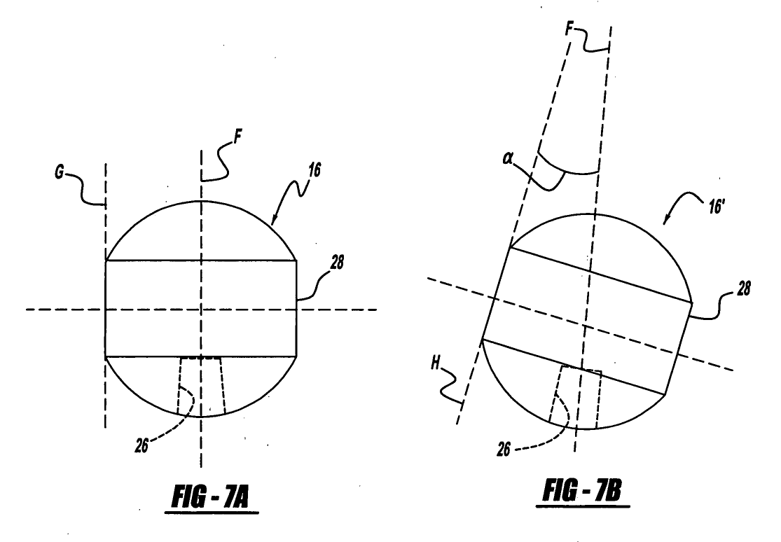

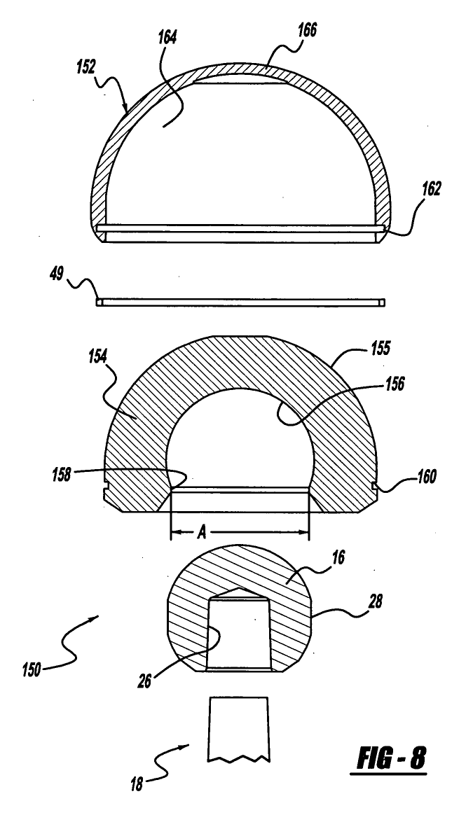

With reference to FIGS. 7A and 7B, as briefly discussed above, the cylindrical equator (CE) 28 of the head 16 can be positioned on the head 16 in any appropriate manner. With reference to FIG. 7A, the head 16 can include the cylindrical equator 28. The female taper 26 can extend a distance into the head 16 and along an axis F. The CE 28 can define an axis or plane G. Plane G can be substantially parallel to the axis F or have an angle of incidence substantially equal to zero. In this way, the CE 28 can be parallel or aligned with the axis F of the head 16.

With reference to FIG. 7B, the head 16′ can also define the CE 28. The head 16′ can define the central axis F that is defined by or extends through the taper 26. The CE 28, however, can define an axis or plane H that includes an angle α relative to the axis G. The angle α can be any appropriate angle such as about 0° to about 90°. For example, the angle α can be about 150. The angle α can be any appropriate angle and can be provided such that the head 16′ includes the CE 28 that is substantially not aligned or not parallel with the axis F.

The angle α or lack of angle can be provided for various reasons, such as assembly reasons and obtaining a selected configuration after positioning the implants for the prosthesis in an anatomy. It will be understood that the angle can be provided in any appropriate manner for achieving a selected result after implantation of the prosthesis. Further, the head 16, 16′ can be positioned on any appropriate implant, such as the femoral stem 18. Thus the angle can also be formed relative to the femoral stem 18. For example, the angle α can be an angle defined between a longitudinal axis of the femoral stem 18 and the plane G.

It will be understood that the CE 28 can be any appropriate shape, dimension, geometry, or the like. The CE 28 can define a passage dimension operable to allow it to pass into a selected portion of a prosthesis, such as including a dimension to allow it to pass through the dimension A or passage of the liner according to various embodiments. Nevertheless, the CE or passage portion 28 can be sized according to various embodiments.

It will be understood that according to various embodiments, a prosthesis can be provided that includes a plurality of features. For example, with reference to FIG. 8, a bipolar prosthesis 150 can be provided. The bipolar prosthesis 150 can include an acetabular shell 152 and a liner portion 154 can be provided to be inserted into the bipolar shell 152. Further, a head, such as the head 16, can be provided to articulate with a selected portion of the liner 154. Finally, as discussed above, the femoral implant 18 can be interconnected with the head 16, such as with the taper 26. The various portions can be any appropriate size and can be selected for various purposes; for example, the head 16 can have a major diameter of about 20 cm to about 80 cm, or any fraction thereof. The head 16 can define the CE 28, as discussed above. Therefore, the head 16 can be inserted in said liner 154 in a selected manner and be held relative thereto due to the geometry of the head 16 relative to the liner 154.

The liner 154 can define a selected geometry and can define an internal arcuate or partial spherical region 156. The arcuate region 156 can substantially define a hemisphere at a selected region and include a portion that extends above the hemisphere such as a constraining or holding portion 158. As discussed above, the constraining ring 40 illustrated in FIG. 6, can be provided to assist in holding the head 16 relative to the liner 154. Nevertheless, the constraining region 158 can be defined by the liner 154 and is substantially integral or formed as one piece or from one piece with the liner 154. The constraining region 158 can be a portion that extends above a hemisphere defined by the internal arcuate portion 156 and can allow for engagement of the head 16 at a selected time.

According to various embodiments, the constraining region 158, or any appropriate constraining region or portion can define the passage dimension A which can be equivalent or allow for passage of the head 16, including the CE or passage portion 28. As discussed above the dimension A can be any appropriate dimension. The constraining region 158, according to various embodiments, can allow access to an internal void via passage into the internal void defined by the liner 154, or any liner according to any of the various embodiments. The liner or any selected portion of the prosthesis can define an internal void that can interact with other portions of the prosthesis, such as allowing for articulation of the head 16 therewith. Nevertheless, the internal void of the prosthesis, including the liner 154, can be any appropriate geometry, size, configuration or the like. Further, the head 16 can be any appropriate geometry, size, configuration or the like.

As discussed above, the head 16 can be aligned such that the CE 28 is able to pass through the constraining region 158 and engage the arcuate region 156 of the liner 154. Once the head 16 moves from the selected or aligned orientation relative to the liner 154, the constraining region 158 can assist in substantially reducing the possibility of dislocation of the head 16 from the liner 154 such as by increasing a lever out force. It will be understood that the head 16 can be first implanted onto the femoral stem 18 prior to its positioning in the liner 154 or according to various assembly procedures, including those discussed herein.

The liner 154 can be positioned within the bipolar shell 152 in any appropriate manner. As discussed above, a locking ring, such as the locking ring 49 can be provided to achieve a selected engagement between the liner 154 and the bipolar shell 152. The liner 154 can define a groove 160 and the bipolar shell 152 and define a complementary groove 162. The locking ring 49 can be provided between the two grooves 160, 162 to substantially hold the liner 154 relative to the bipolar shell 152 in any appropriate manner, such as those discussed above.

The bipolar shell 152 defines an internal cavity 164 that can interact in a selected manner with the liner 154. For example, a substantially arcuate portion of the internal region 164 can engage an external surface 155 of the liner 154. The bipolar shell 152 can also define an exterior surface 166 that can define any appropriate arcuate portion. The exterior surface 166 can be provided to articulate with the selected portion of the anatomy. For example, the bipolar shell 152 can be positioned relative to a natural or unprepared acetabulum 76 of a patient.

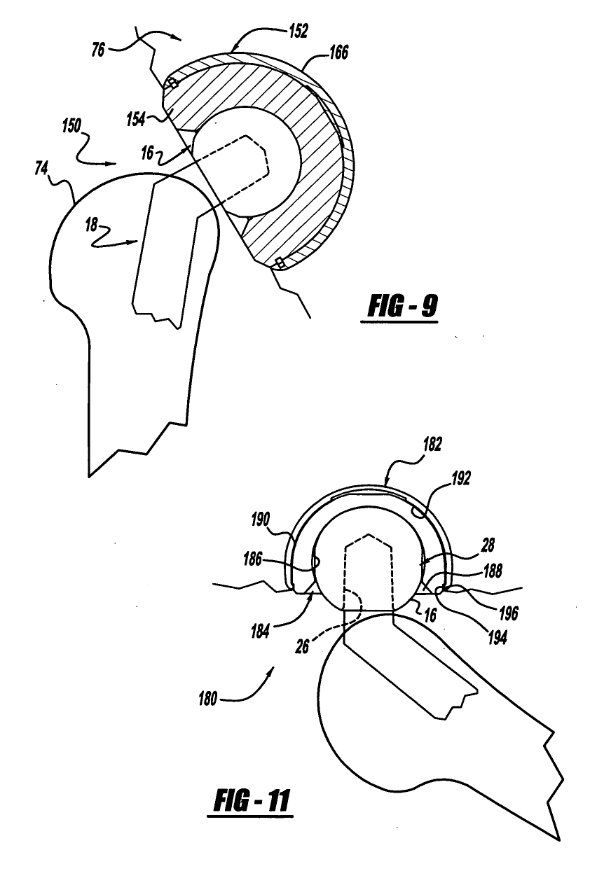

With reference to FIG. 9, the bipolar shell 152 can articulate with the acetabulum 76. The exterior surface 166 of the bipolar shell 152 need not be permanently fixed to the acetabulum 76 of a patient. Rather the exterior surface 166 can be provided to articulate in the acetabulum in any appropriate manner. Therefore, the acetabulum need not be prepared in any specific manner, such as reaming, implanting or affixing a shell, or the like. Rather the bipolar shell 152 can articulate with the acetabulum 76 in a selected manner.

Further, the head 16 can articulate within the liner 154 as well. The head 16 can articulate within the liner 154 in any appropriate manner, such as achieving a substantially anatomical movement after implantation of the prosthesis 150. Therefore, both the head 16 can articulate with the liner 154 and the shell 152 can articulate with the acetabulum 76 to achieve a bipolar articulation.

Both the bipolar shell 152 and the liner 154 can be formed of any appropriate materials. For example, the bipolar shell 152 can be formed of a selected metal or metal alloy or of a polymer material. For example, the bipolar shell 152 can be formed of a cobalt chromium alloy to articulate with the natural acetabulum 76. Alternatively, the bipolar shell 152 can be formed of a polymer material to articulate with the natural acetabulum 76. Similarly, the liner 154 can be formed of a similar or a different material than the bipolar shell 152. Regardless, the materials can be selected to achieve a selected result, such as a longevity, a coefficient of friction, or the like. It will be understood, however, that the materials can be any appropriate materials and selected for various purposes.

Nevertheless, the CE 28 can be provided on the head 16 to assist in positioning the head 16 relative to the liner 154. The head 16, can resist dislocation from the liner 154 after implantation of the prosthesis 150. Further, the prosthesis can be reduced even if the head 16 becomes dislocated from the liner 154 by simply realigning the CE 28 with the opening of the liner 154 such that the CE 28 passes through the restraining portion 158 of the liner 154, as discussed above. This can allow for a closed reduction of the prosthesis 150.

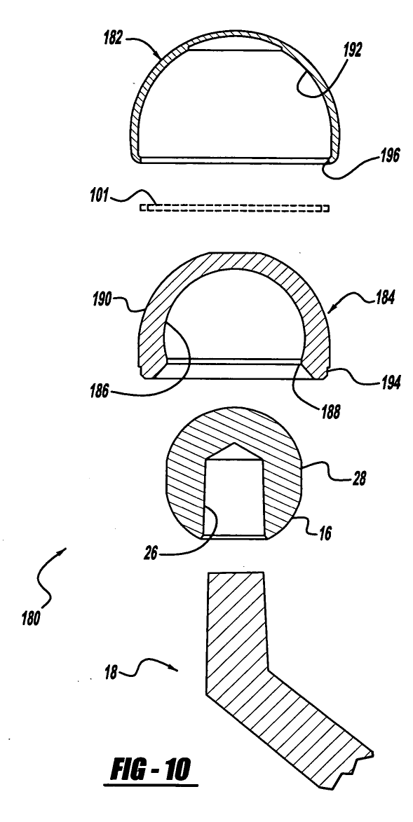

With reference to FIG. 10, a prosthesis 180 is illustrated. The prosthesis 180 can include the head 16 that defines the CE 28, as discussed above. Further, the head 16 can define a recess 26 for engaging or interconnecting with a femoral component 18. It will be understood that any appropriate interconnection can be provided according to various embodiments, and the recess or taper 26 is merely exemplary.

The prosthesis 180 can further include a bipolar shell 182 and a liner 184. The liner 184 can define an internal cavity or arcuate region 186. The internal arcuate region 186 can define a portion or a hemisphere and also include a constraining portion 188 that extends above the hemisphere. The constraining portion 188 can be substantially similar to the constraining portion 158 illustrated in FIG. 8. Further, the constraining region 188 can be integral or formed with the liner 184 or can include an external portion, such as the constraining ring 40. Regardless, the constraining region 188 can interact with the head 16 to assist in holding the head 16 relative to the liner 184, according to various embodiments.

The liner 184 can further define an exterior arcuate portion 190. The exterior arcuate portion 190 can include a portion that substantially engages or mates with an internal arcuate portion 192 of the bipolar shell 182. Although the exterior portion 190 of the liner 184 need not contact the entire interior region 192 of the shell 182, it can be provided to contact at least a portion thereof to distribute a force. Further, the liner 184 can define a depression or notch 194 substantially around a circumference or portion of the circumference of the liner 184. The depression 194 can be engaged by a projection 196 defined by the bipolar shell 182. The projection 196 can engage the liner 184 in any appropriate manner, such as a friction fit therewith. As the liner 184 is forced into the shell 182, the projection 196 can move along the exterior surface 190 of the liner 184 until it reaches the depression 194 and moves into the depression 194. Therefore, the shell 182 may be provided to deflect a certain amount or the liner 184 may be formed to deflect or compress a certain amount to allow the projection 196 to pass over a region to move into the depression 194.

The projection 196 can serve a purpose similar to the locking ring 49 according to various embodiments. Therefore, the projection 196 can substantially hold the liner 184 relative to the bipolar shell 182 for various purposes, such as holding the liner 184 in a selected position during an anatomical movement of the prosthesis 180. It will be understood that the liner 184 can be assembled with the shell 182 at any appropriate time, such as prior to delivery to a user or by a user. The integral or monolithic projection 196 can allow for a selected or increased holding force of the liner 184 relative to the bipolar shell 182. It will be understood that a locking ring 101, illustrated in phantom, can be provided in the alternative of the projection 196. The locking ring 101 can work substantially similarly to the locking ring 49, discussed above. Therefore, it will be understood that the liner 184 can be interconnected with the bipolar shell 182 according to various embodiments.

It will be understood that the bipolar shell 182 can articulate within the acetabulum 76 similar to the bipolar shell 152. Therefore, the head 16 can articulate within the liner 184 and the bipolar shell 182 can articulate within the acetabulum 76 to allow for a substantially bipolar articulation of the prosthesis 180. Nevertheless, the head 16 can be positioned relative to the liner 184 by aligning the CE 28 with the opening defined by the holding region 188 to allow for positioning of the head 16 within the liner 184 and then a displacement of the head 16 relative to the liner 184 can allow for a substantial force that can resist dislocation of the head 16 from the liner 184. As discussed above, the various portions, such as the CE 28, can allow for ease of implantation, closed reduction, and various other purposes.

It will be understood that the various portions of the various embodiments can be provided together or separately for various purposes. For example, the prosthesis 180 can be positioned relative to the anatomy, such as the femur 74 and the acetabulum 76. Although the bipolar shell 182 can be provided to substantially articulate with the acetabulum 76, the acetabular shell 182 can also be substantially fixed relative to the acetabulum 76, according to various techniques such as screws, cement, or the like. Therefore, it will be understood that while the bipolar shell 182 can be provided to articulate within the acetabulum 76, it can also be provided to be substantially fixed relative to the acetabulum 76. Further, the locking ring 101 illustrated in phantom and FIGS. 10 and 11, can also be provided in addition to the projection 196, alternatively to the projection 196 or according to any appropriate combination. Therefore, the prostheses, including the prosthesis 180, according to various embodiments, can include selected portions or features as discussed above.

It will be understood that the materials of the various embodiments can be any appropriate materials. For example, the bipolar shells, or the shells according to any various embodiments, can be formed of any appropriate materials. For example, a hard material, such as a metal, a metal alloy, a diamond material, a ceramic, a diamond coated material, or the like, can be provided to form the shell. The shell can be formed to include a smooth exterior to substantially articulate with the acetabulum 76 in a selected manner, such as a non-abrasive manner. Further, the liners, according to various embodiments, can be provided of selected materials including ceramics, metals or metal alloys, polymers, or the like. Similarly, the head 16 can be formed of any appropriate material, such as ceramics, metals or metal alloys, polymers or the like. It will be understood that each of the various components can be formed of different materials or formed of the same materials to achieve a selected result. Nevertheless, the configurations can allow for a selected implantation, a selected resistance to dislocation, the ability for a close reduction, or other features.

As discussed, according to various embodiments, the dimension A can define a passage dimension of the liner or a selected portion of the prosthesis to allow for passage of the head 16 into an interior portion of a selected portion of the prosthesis, such as the liner. It will be understood according to various embodiments that the dimension A can be equivalent to any appropriate dimension of the head 16, such as the cylindrical equator dimension 28. It will be understood that the dimension of the CE 28, the dimension of A, can be any appropriate dimension and can be selected according to various embodiments. Nevertheless, the dimension A and a diameter of the CE 28 can be equivalent to allow for passage to the head 16 into a selected portion of the prosthesis, such as an internal portion of the liner. Further, as discussed above and herein, the CE 28 can allow for passage of the head 16 into a selected portion of the prosthesis and for movement of the head 16 to a different orientation that can allow for resisting dislocation of the head 16 from the prosthesis. It will be understood that according to various embodiments, including a bipolar feature, and can include the head 16 that resisted dislocation from the selected portion of the prosthesis.

The teachings are merely exemplary in nature and, thus, variations that do not depart from the gist of the teachings are intended to be within the scope of the teachings. Such variations are not to be regarded as a departure from the spirit and scope of the teachings.

What is claimed is:

1. A prosthesis for replacement of a natural joint in an anatomy that can resist dislocation, the prosthesis comprising:
a liner defining an internal void portion defining an internal void dimension, and defining a constraining opening having a passage dimension smaller than the internal void dimension;
a head portion having a head dimension complementary to the internal void dimension;
wherein the head portion includes a passage portion having a dimension similar to the passage dimension;
wherein the head portion is operable to be implanted into the internal void portion during an operative procedure.

2. The prosthesis of claim 1, further comprising:
a shell portion positionable in a first bony portion of the joint relative to the head portion;
wherein the shell portion is operable to articulate relative to the anatomy and the head portion is operable to articulate relative to the liner portion;
wherein the liner is positionable within the shell.

3. The prosthesis of claim 2, wherein the liner is fixed relative to the shell portion.

4. The prosthesis of claim 2, wherein the liner is locked relative to the shell portion.

5. The prosthesis of claim 4, wherein the liner is locked with a locking ring, a shell projection, or combinations.

6. The prosthesis of claim 2, further comprising:
a stem operable to be positioned in a second bony portion of the anatomy;
wherein the stem is operable to be interconnected with the head portion to allow for movement of the second bony portion relative to the first bony portion of the anatomy.

7. The prosthesis of claim 1, wherein the passage portion of the head portion defines a cylindrical diameter.

8. The prosthesis of claim 6, wherein the passage portion is alignable with the constraining opening in substantially only one insertable orientation, wherein the insertable orientation includes a substantially unnatural orientation of the second bony portion relative to the first bony portion.

9. The prosthesis of claim 1, wherein the head portion defines a first axis and the passage portion defines a second axis:
wherein the second axis of the passage portion is formed at an angle relative to the first axis of the head portion.

10. The prosthesis of claim 1, further comprising:
a shell operable to contact a selected portion of the liner;
a stem operably extending from the head portion;
wherein the shell, the liner, the head portion, and the stem are each provided as a separate component.

11. The prosthesis of claim 1, wherein the passage portion of the head portion is operable to allow a selected flow of fluid through the constraining opening into the internal void of the liner.

12. A prosthesis for replacement of a natural joint in an anatomy, the prosthesis comprising:
a first member having an internal wall defining an internal void having an internal void dimension;
a second member having a second dimension and defining a passage portion and operable to articulate with the internal wall;
a third member operable to be fixed relative to the first member and articulate with the anatomy;
wherein the second member is adapted to be implanted in the first member relative to the internal wall during an operative procedure;
wherein the passage portion is operable to substantially ensure contact with less than the entire internal wall.

13. The prosthesis of claim 12, wherein the first member includes a constraining portion operable to resist removal of the second member from the internal void after implantation of the second member within the first member.

14. The prosthesis of claim 13, wherein the constraining portion includes a monolithic wall, a constraining ring, a constraining projection, or combinations thereof.

15. The prosthesis of claim 12, wherein the second member defines a portion of a sphere and the passage portion is positioned at a selected equator of a portion of the sphere that includes a radius less than a radius defined at a portion of the sphere not defining the passage portion.

16. The prosthesis of claim 12, wherein the passage portion is operable to allow a flow of fluid into the internal void of the first member.

17. The prosthesis of claim 12, further comprising:
a fourth member;
wherein the fourth member is operable to be positioned in a portion of the anatomy operable to move relative to the portion of the anatomy where the first member is positioned;
wherein movement of the fourth member is operable to cause articulation of the second member with the first member and the third member with the anatomy.

18. The prosthesis of claim 12, wherein the second member resists dislocation from its position within the internal void of the first member after implantation of the second member into the first member.

19. A method of positioning a prosthesis relative to a selected boney portion to replace a natural joint, the method comprising:
positioning a first member relative to the boney portion and the first member defining an internal void and a passable portion defining a passage dimension;
positioning a second member relative to a boney portion defining a first dimension operable to be positioned relative to the internal void and defining a passage portion defining a passage dimension having a selected orientation relative to the second member and operable to pass through the passable portion, wherein the passage dimension is less than the first dimension;
orienting the second member such that the passage portion is generally aligned with the passage portion;
positioning the second member in the internal void;
wherein the second member is substantially held within the internal void.

20. The method of claim 19, further comprising:
constraining the second member in the first member with a constraining member; a monolithic constraining portion, or combinations thereof.

21. The method of claim 19, further comprising:
a third member operable to articulate with the anatomy;
holding the first member in a selected position to third member.

22. The method of claim 21, wherein holding the first member in a selected position to third member includes locking the first member to the third member with a locking ring, locking the first member to the third member with a locking projection, or combinations thereof.

23. The method of claim 21, further comprising:
articulating the second member with the first member and articulating the third member with the anatomy.

24. The method of claim 21, further comprising:
providing a fourth member;
implanting the fourth member into a bone portion of the anatomy;
interconnecting the second member with the fourth member.

25. The method of claim 24, wherein the first member is an acetabular shell liner, the second member is a femoral head replacement; the third member is an acetabular shell, and the further member is a femoral stem.

26. The method of claim 19, further comprising:
positioning the first member, the second member, or combinations thereof relative to the anatomy.

27. The method of claim 26, further comprising:
positioning the second member in the internal void prior to
positioning the first member, the second member, or combinations thereof relative to the anatomy.
